(19) United States
(12) Patent Application Publication
(10) Pub. No.: US 2012/0046185 A1
(43) Pub. Date: Feb. 23, 2012

(54) PANEL OF BIOMARKERS FOR OVARIAN CANCER

(75) Inventors: Daniel W. Chan, Clarksville, MD (US); Zhen Zhang, Dayton, MD (US); Eric Fung, Los Altos, CA (US)

(73) Assignees: Vermillion, Inc., Austin, TX (US); The Johns Hopkins University, Baltimore, MD (US)

(21) Appl. No.: 13/204,566

(22) Filed: Aug. 5, 2011

Related U.S. Application Data
(60) Provisional application No. 61/371,411, filed on Aug. 6, 2010.

(51) Int. Cl. C40B 30/04; H01J 49/26; C07K 16/18; G01N 33/574; C07K 16/30

(52) U.S. Cl. 506/9; 436/501; 530/387.9; 250/282

(57) ABSTRACT

The present invention provides a panel of protein-based biomarkers that are useful in diagnosing ovarian cancer in a subject. In particular, the panel of biomarkers of the invention are useful to classify a subject sample as having ovarian cancer or non-ovarian cancer.

### CROSS-REFERENCE TO RELATED APPLICATIONS

This application claims the benefit of U.S. Provisional Application Ser. No. 61/371,411, filed Aug. 6, 2010, the contents of which are incorporated herein by reference in its entirety.

### BACKGROUND OF THE INVENTION

Ovarian cancer is among the most lethal gynecologic malignancies in developed countries. Annually in the United States alone, approximately 23,000 women are diagnosed with the disease and almost 14,000 women die from it. Despite progress in cancer therapy, ovarian cancer mortality has remained virtually unchanged over the past two decades. Given the steep survival gradient relative to the stage at which the disease is diagnosed, early detection remains the most important factor in improving long-term survival of ovarian cancer patients.

Ovarian tumors are being detected with increasing frequency in women of all ages, yet there is no standardized or reliable method to determine which are malignant prior to surgery. In 1994, the National Institutes of Health (NIH) released a consensus statement indicating that women with ovarian masses having been identified preoperatively as having a significant risk of ovarian cancer should be given the option of having their surgery performed by a gynecologic oncologist. At present, the National Comprehensive Cancer Network (NCCN), the Society of Gynecologic Oncologists (SGO), SOGC clinical practice guidelines, Standing Subcommittee on Cancer of the Medical Advisory Committee, and several other published statements, all recommend that women with ovarian cancer be under the care of a gynecologic oncologist (GO).

Recent publications on breast, bladder, gastrointestinal, and ovarian cancers have reported improved outcome when cancer management involves a surgical specialist. In addition, a recent meta-analysis of 18 ovarian cancer studies found that the early involvement of a gynecologic oncologist, rather than a general surgeon or general gynecologist, improved patient outcomes. The authors concluded: 1) subjects with early stage disease are more likely to have comprehensive surgical staging, facilitating appropriate adjuvant chemotherapy, 2) subjects with advanced disease are more likely to receive optimal cytoreductive surgery, and 3) subjects with advanced disease have an improved median and overall 5-year survival. Despite the availability of this important information, only a fraction of women with malignant ovarian tumors (an estimated 33%) are referred to a gynecologic oncologist for the primary surgery. Based on reported patterns of care for ovarian cancer management, the majority of women in the United States may not be receiving optimal care for this disease.

The decision for operative removal of an ovarian tumor, and whether a generalist or specialist should perform the surgery, is based on interpretations of physical examination, imaging studies, laboratory tests, and clinical judgment. Pelvic examination alone is inadequate to reliably detect or differentiate ovarian tumors, particularly in early stages when ovarian cancer treatment is most successful. Examination has also been eliminated from the Prostate, Lung, Colorectal and Ovarian cancer screening trial algorithm. Pelvic ultrasound is clinically useful and the least expensive imaging modality, but has limitations in consistently identifying malignant tumors. In general, nearly all unilocular cysts are benign, whereas complex cystic tumors with solid components or internal papillary projections are more likely to be malignant. CA 125 has been used alone or in conjunction with other tests in an effort to establish risk of malignancy. Unfortunately, CA 125 has low sensitivity (50%) in early stage ovarian cancers, and low specificity resultant from numerous false positives in both pre- and postmenopausal women.

The American College of Obstetrics and Gynecology (ACOG) and the SGO have published referral guidelines for patients with a pelvic mass. These guidelines include: patient age, serum CA 125 level, physical examination, imaging results, and family history. This referral strategy has been evaluated both retrospectively and prospectively. In a single institution review, Dearking and colleagues concluded that the guidelines were useful in predicting advanced stage ovarian cancer, but “performed poorly in identifying early-stage disease, especially in premenopausal women, primarily due to lack of early markers and signs of ovarian cancer”.

Thus, it is desirable to have a reliable and accurate method of determining the ovarian cancer status in patients, the results of which can then be used to manage subject treatment.

### BRIEF SUMMARY OF THE INVENTION

The present invention provides methods and kits that are useful for preoperative assessment of ovarian tumors. The measurement of the panel of biomarkers set forth herein in patient samples provides information that diagnosticians can use to assess an ovarian tumor and determine if the tumor is malignant or benign. In embodiments, the markers are identified and quantified by immunoassay.

More specifically, the biomarker panel of the invention comprises five polypeptides and fragments thereof as set forth in Table 1. These biomarkers are CA 125, transthyretin (prealbumin), apolipoprotein A1, β-2-microglobulin, and transferrin.

In aspects, the invention provides methods for identifying ovarian cancer status in a subject. In embodiments, the methods involve determining the level of biomarkers in a biological sample from the subject, wherein the biomarkers comprise β-2-microglobulin, CA 125, transthyretin (prealbumin), apolipoprotein A1, transferrin, fragments thereof, or a combination thereof. In embodiments, the methods involve comparing the level of the biomarkers to a reference. In embodiments, the subject is identified as having ovarian cancer when: i) there is an increase in the amount of β-2-microglobulin or a fragment thereof, ii) there is an increase in the amount of CA 125 or a fragment thereof, iii) there is a decrease in the amount of transthyretin (prealbumin) or a fragment thereof, iv) there is a decrease in the amount of apolipoprotein A1 or a fragment thereof, v) there is a decrease in the amount of transferrin or a fragment thereof relative to the reference, or vi) a combination thereof.

In aspects, the invention provides methods for detecting ovarian cancer or early stage ovarian cancer in a subject. In embodiments, the methods involve determining the level of biomarkers in a biological sample from the subject, wherein the biomarkers comprise β-2-microglobulin, CA 125, transthyretin (prealbumin), apolipoprotein A1, transferrin, fragments thereof, or a combination thereof. In embodiments, the methods involve comparing the level of the biomarkers to a reference. In embodiments, the subject is identified as having ovarian cancer or early stage ovarian cancer when: i) there is an increase in the amount of β-2-microglobulin or a fragment thereof, ii) there is an increase in the amount of CA 125 or a fragment thereof, iii) there is a decrease in the amount of transthyretin (prealbumin) or a fragment thereof, iv) there is a decrease in the amount of apolipoprotein A1 or a fragment thereof, v) there is a decrease in the amount of transferrin or a fragment thereof relative to the reference, or vi) a combination thereof. In related embodiments, the early stage ovarian cancer is stage I ovarian cancer or stage II ovarian cancer.

In aspects, the invention provides methods for monitoring ovarian cancer therapy in a subject. In embodiments, the methods involve determining the level of biomarkers in a biological sample from the subject, wherein the biomarkers comprise β-2-microglobulin, CA 125, transthyretin (prealbumin), apolipoprotein A1, transferrin, fragments thereof, or a combination thereof. In embodiments, the methods involve comparing the level of the biomarkers to a reference. In embodiments, a therapy that i) decreases the amount of β-2-microglobulin or a fragment thereof, ii) decreases the amount of CA 125 or a fragment thereof, iii) increases the amount of transthyretin (prealbumin) or a fragment thereof, iv) increases the amount of apolipoprotein A1 or a fragment thereof, v) increases the amount of transferrin or a fragment thereof relative to the reference is identified as effective, or vi) a combination thereof is effective.

In any of the above aspects, the methods further involve managing subject treatment based on the status. In embodiments, the subject is treated with surgery, radiotherapy, chemotherapy, or a combination thereof, if the subject is identified as having ovarian cancer or if the therapy is identified as ineffective. In related embodiments, the surgery is performed by a gynecologic oncologist.

In any of the above aspects, the reference is a control. In embodiments, the control is obtained from a patient having ovarian cancer. In embodiments, the reference is obtained from the subject prior to therapy or at an earlier time point during therapy.

In any of the above aspects, the methods further involve managing subject treatment based on the status.

In aspects, the invention provides methods for selecting a treatment for a subject diagnosed as being at risk of having ovarian cancer. In embodiments, the methods involve determining the level of biomarkers in a biological sample from the subject, wherein the biomarkers comprise β-2-microglobulin, CA 125, transthyretin (prealbumin), apolipoprotein A1, transferrin, fragments thereof, or a combination thereof. In embodiments, the methods involve comparing the level of the biomarkers to a reference. In embodiments, the methods involve selecting a treatment selected from the group consisting essentially of: surgery, chemotherapy, radiotherapy, and a combination thereof, if the level of the biomarkers is altered relative to the reference. In related embodiments, the surgery is performed by a gynecologic oncologist. In embodiments, the treatment is selected when i) there is an increase in the amount of β-2-microglobulin or a fragment thereof, ii) there is an increase in the amount of CA 125 or a fragment thereof, iii) there is a decrease in the amount of transthyretin (prealbumin) or a fragment thereof, iv) there is a decrease in the amount of apolipoprotein A1 or a fragment thereof, v) there is a decrease in the amount of transferrin or a fragment thereof relative to the reference, or vi) a combination thereof.

In any of the above aspects, the level of the biomarkers is determined by any method well known in the art, including, but not limited to, the detection methods described herein. In embodiments, the level of the biomarkers is determined by immunoassay, biochip array, mass spectrometry, or a combination thereof. In related embodiments, the biochip array is a protein biochip array.

In any of the above aspects, the subject is further evaluated one or more additional diagnostic procedures. In embodiments, the subject is further evaluated by medical imaging, physical exam, laboratory test(s), menopausal status, clinical history, family history, gene test, BRCA test, and the like. Medical imaging is well known in the art. As such, the medical imaging can be selected from any well known method of imaging, including, but not limited to, ultrasound, computed tomography scan, positron emission tomography, photon emission computerized tomography, and magnetic resonance imaging.

In any of the above aspects, the sample can be any biological sample suitable for anaylsis. In embodiments, the biological sample can be blood, blood serum, plasma, saliva, urine, ascites, cyst fluid, a homogenized tissue sample (e.g., a tissue sample obtained by biopsy), a cell isolated from a patient sample, and the like. In embodiments, the biological sample is blood, blood serum, plasma. In related embodiments, the biological sample is serum.

In any of the above aspects, the subject is premenopausal.

In any of the above aspects, the subject is postmenopausal.

In any of the above aspects, comparing the level of the biomarkers to a reference is performed by a software classification algorithm.

In aspects, the invention provides kits for aiding the diagnosis of ovarian cancer, monitoring the treatment of ovarian cancer, or for identifying a course of treatment for cancer. In embodiments, the kits contain one or more agents capable of detecting or capturing β-2-microglobulin, CA 125, transthyretin (prealbumin), apolipoprotein A1, transferrin, or a combination thereof. In embodiments, the kits further contain instructions for using the agent(s) to detect β-2-microglobulin, CA 125, transthyretin (prealbumin), apolipoprotein A1, transferrin, or a combination thereof. In embodiments, the instructions describe using the agent(s) in any of the methods described herein.

In aspects, the agent is an antibody. In embodiments, the antibody specifically binds to β-2-microglobulin, CA 125, transthyretin (prealbumin), apolipoprotein A1, transferrin, or fragments thereof.

In aspects, the agent is labeled. In embodiments, the kit comprises agent(s) for detecting the label. The label can be any label well known in the art including, but not limited to, radiolabels, fluorescent labels, and imaging agents.

In aspects, the kit further comprises one or more control samples. In embodiments, the control samples contain β-2-microglobulin, CA 125, transthyretin (prealbumin), apolipoprotein A1, transferrin, or a combination thereof.

In any of the above aspects, the methods involve determining the level of β-2-microglobulin, CA 125, transthyretin (prealbumin), apolipoprotein A1, transferrin, and fragments thereof. In related embodiments, a subject is identified, therapy is determined effective, or treatment is selected when i) there is an increase in the amount of β-2-microglobulin or a fragment thereof, ii) there is an increase in the amount of CA 125 or a fragment thereof, iii) there is a decrease in the amount of transthyretin (prealbumin) or a fragment thereof, and iv) there is a decrease in the amount of apolipoprotein A1 or a fragment thereof, v) there is a decrease in the amount of transferrin or a fragment thereof relative to the reference.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### Definitions

To facilitate an understanding of the present invention, a number of terms and phrases are defined below.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “biomarker” as used herein generally refers to a molecule that is differentially present in a sample taken from a subject of one phenotypic status (e.g., having a disease) as compared with another phenotypic status (e.g., not having the disease). A biomarker is differentially present between different phenotypic statuses if the mean or median level of the biomarker in a first phenotypic status relative to a second phenotypic status is calculated to represent statistically significant differences. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. As such, biomarkers can find use as markers for, for example, disease (diagnostics), therapeutic effectiveness of a drug (theranostics), and of drug toxicity.

By “agent” is meant any small molecule chemical compound, antibody, nucleic acid molecule, or polypeptide, or fragments thereof.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The term “ovarian cancer” refers to both primary ovarian tumors as well as metastases of the primary ovarian tumors that may have settled anywhere in the body.

The term “ovarian cancer status” refers to the status of the disease in the patient. Examples of types of ovarian cancer statuses include, but are not limited to, the subject's risk of cancer, the presence or absence of disease, the stage of disease in a patient, and the effectiveness of treatment of disease. In embodiments, a subject identified as having a pelvic mass is assessed to identify if their ovarian cancer status is benign or malignant.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

As used herein, the term “sample” includes a biologic sample such as any tissue, cell, fluid, or other material derived from an organism.

By “reference” is meant a standard of comparison. For example, the biomarker level(s) present in a patient sample may be compared to the level of the compound(s) in a corresponding healthy cell or tissue or in a diseased cell or tissue (e.g., a cell or tissue derived from a subject having ovarian cancer).

By “specifically binds” is meant a compound (e.g., antibody) that recognizes and binds a molecule (e.g., polypeptide), but which does not substantially recognize and bind other molecules in a sample, for example, a biological sample.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cutpoints of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test. In embodiments, biomarkers and diagnostic methods of the present invention have an AUC greater than 0.50, greater than 0.60, greater than 0.70, greater than 0.80, or greater than 0.9.

Other useful measures of the utility of a test are positive predictive value (“PPV”) and negative predictive value (“NPV”). PPV is the percentage of actual positives who test as positive. NPV is the percentage of actual negatives that test as negative.

As described in detail herein, any method well known in the art can be used to measure a panel of biomarkers. In aspects of the invention, the panel of biomarkers are measured using any immunoassay well known in the art. In embodiments, the immunoassay can be, but is not limited to, ELISA, western blotting, and radioimmunoassay.

In embodiments, the panel of biomarkers described herein are measured using a biochip array. Biochip arrays useful in the invention include protein and nucleic acid arrays. One or more markers are captured on the biochip array and subjected to laser ionization to detect the molecular weight of the markers. Analysis of the markers is, for example, by molecular weight of the one or more markers against a threshold intensity that is normalized against total ion current. In embodiments, logarithmic transformation is used for reducing peak intensity ranges to limit the number of markers detected.

In aspects of the invention, the panel of biomarkers are measured using laser desorption/ionization mass spectrometry, comprising providing a probe adapted for use with a mass spectrometer comprising an adsorbent attached thereto, and contacting the subject sample with the adsorbent, and; desorbing and ionizing the marker or markers from the probe and detecting the deionized/ionized markers with the mass spectrometer.

In embodiments, the laser desorption/ionization mass spectrometry comprises: providing a substrate comprising an adsorbent attached thereto; contacting the subject sample with the adsorbent; placing the substrate on a probe adapted for use with a mass spectrometer comprising an adsorbent attached thereto; and, desorbing and ionizing the marker or markers from the probe and detecting the desorbed/ionized marker or markers with the mass spectrometer.

The adsorbent can for example be hydrophobic, hydrophilic, ionic or metal chelate adsorbent, such as, nickel or an antibody, single- or double stranded oligonucleotide, amino acid, protein, peptide or fragments thereof.

In aspects of the invention, the step of correlating the measurement of the biomarkers with ovarian cancer status is performed by a software classification algorithm.

The methods of the present invention can be performed on any type of patient sample that would be amenable to such methods, e.g., blood, serum, plasma, and the like.

The present invention also provides kits comprising (a) reagents that bind the panel of biomarkers set forth in Table 1; and, optionally, (b) a container comprising at least one of the biomarkers. While the reagents can be any type of reagent, in embodiments, the reagents are antibodies specific for each of the biomarkers. In related embodiments, the kit comprises five antibodies, each specific for one of the biomarkers of the panel of biomarkers set forth in Table 1, and instructions for use.

Certain kits of the present invention further comprise a wash solution that selectively allows retention of the bound biomarker to the capture reagent as compared with other biomarkers after washing.

Measurement of the protein biomarkers using the kit can be done by any method well known in the art, including, but not limited to, mass spectrometry or immunoassay, e.g., an ELISA.

Purified proteins for detection of ovarian cancer are also provided for. Purified proteins include a purified peptide of any of the biomarkers set forth in Table 1. The invention also provides this purified peptide further comprising a detectable label.

The kits of the invention may further comprise one or more purified biomarkers to be used as standards to determine if a biomarker is under or over expressed.

In another embodiment, non-invasive medical imaging techniques such as transvaginal ultrasound, positron emission tomography (PET) or single photon emission computerized tomography (SPECT) imaging are particularly useful for the detection of a tumor. Once a tumor, e.g., a pelvic tumor, has been identified, the methods and kits of the invention can be used to determine if the tumor is malignant or benign and to determine a course of treatment.

Other aspects of the invention are described infra.

### BRIEF DESCRIPTION OF THE DRAWINGS

FIG. 1 includes a graph showing the receiving-operator-characteristic (ROC) curve analysis of the panel of biomarkers described herein in the preoperative risk of malignancy assessment for ovarian tumors in pre- and postmenopausal women.

FIG. 2 sets forth the amino acid sequence of β-2-microglobulin (SwissProt Accession Number P61769) (SEQ ID NO: 1).

FIG. 3 sets forth the amino acid sequence of CA 125 (SwissProt Accession Number Q8WXI7) (SEQ ID NO: 2).

FIG. 4 sets forth the amino acid sequence of transthyretin (prealbumin) (SwissProt Accession Number P02766) (SEQ ID NO: 3).

FIG. 5 sets forth the amino acid sequence of apolipoprotein A1 (SwissProt Accession Number P02647) (SEQ ID NO: 4).

FIG. 6 sets forth the amino acid sequence of transferrin (SwissProt Accession Number Q06AH7) (SEQ ID NO: 5).

### DETAILED DESCRIPTION OF THE INVENTION

### 1. Introduction

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease.

### 2. Biomarkers for Ovarian Cancer

2.1. Biomarkers

This invention provides a panel of polypeptide biomarkers that are differentially present in subjects having ovarian cancer, in particular, a benign vs. malignant pelvic mass. The biomarkers of this invention are differentially present depending on ovarian cancer status, including, subjects having ovarian cancer vs. subjects that do not have ovarian caner.

The biomarker panel of the invention is presented in the following Table 1.

TABLE 1

| | Up or down |
|---|---|
| | regulated in |
| Biomarker | ovarian cancer |
| CA 125 | UP |
| Transthyretin | DOWN |
| (prealbumin) | |
| Apolipoprotein | DOWN |
| β-2 microglobulin | UP |
| transferrin | DOWN |

As would be understood, references herein to a biomarker of Table 1, a panel of biomarkers, or other similar phrase indicates the five biomarkers as set forth in the above Table 1.

In aspects of the invention, the biological source for detection of the biomarkers is serum. However, in embodiments, the biomarkers can be detected in other biological samples, including, but not limited to, blood, blood serum, plasma, saliva, urine, ascites, cyst fluid, a homogenized tissue sample (e.g., a tissue sample obtained by biopsy), a cell isolated from a patient sample, and the like.

The biomarkers of this invention are biomolecules. Accordingly, this invention provides these biomolecules in isolated form. The biomarkers can be isolated from biological fluids, such as urine or serum. They can be isolated by any method known in the art, based on both their mass and their binding characteristics. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography.

2.2. β-2 Microglobulin

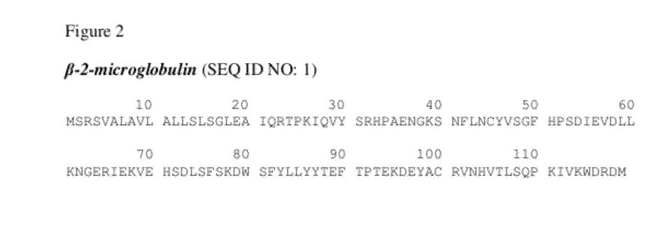

One exemplary biomarker that is useful in the methods of the present invention is β2-microglobulin. β2-microglobulin is described as a biomarker for ovarian cancer in U.S. provisional patent publication 60/693,679, filed Jun. 24, 2005 (Fung et al.). The mature form of β2-microglobulin is a 99 amino acid protein derived from an 119 amino acid precursor (GI:179318; SwissProt Accession No. P61769). The amino acid sequence of β-2-microglobulin is set forth in FIG. 2 (SEQ ID NO: 1). The mature form of β-2-microglobulin consist of residues 21-119 pf SEQ ID NO: 1. β2-microglobulin is recognized by antibodies. Such antibodies can be made using any method well known in the art, and can also be commercially purchased from, e.g., Abcam (catalog AB759) (www.abcam.com, Cambridge, Mass.). In aspects of the invention, β2-microglobulin is upregulated in subjects with ovarian cancer as compared to subjects that do not have ovarian cancer.

2.3 CA 125

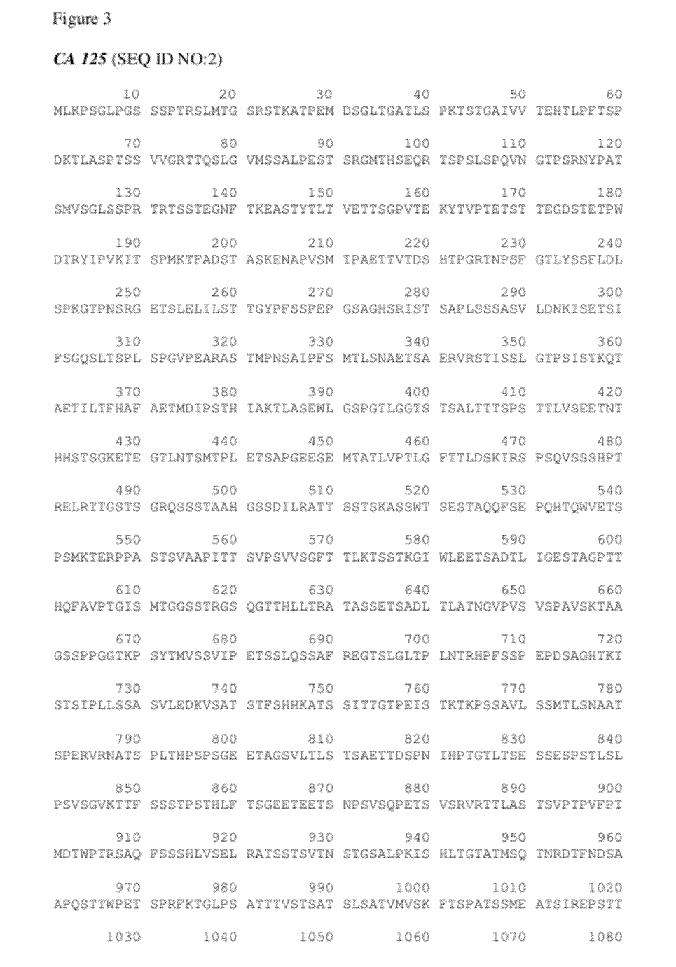

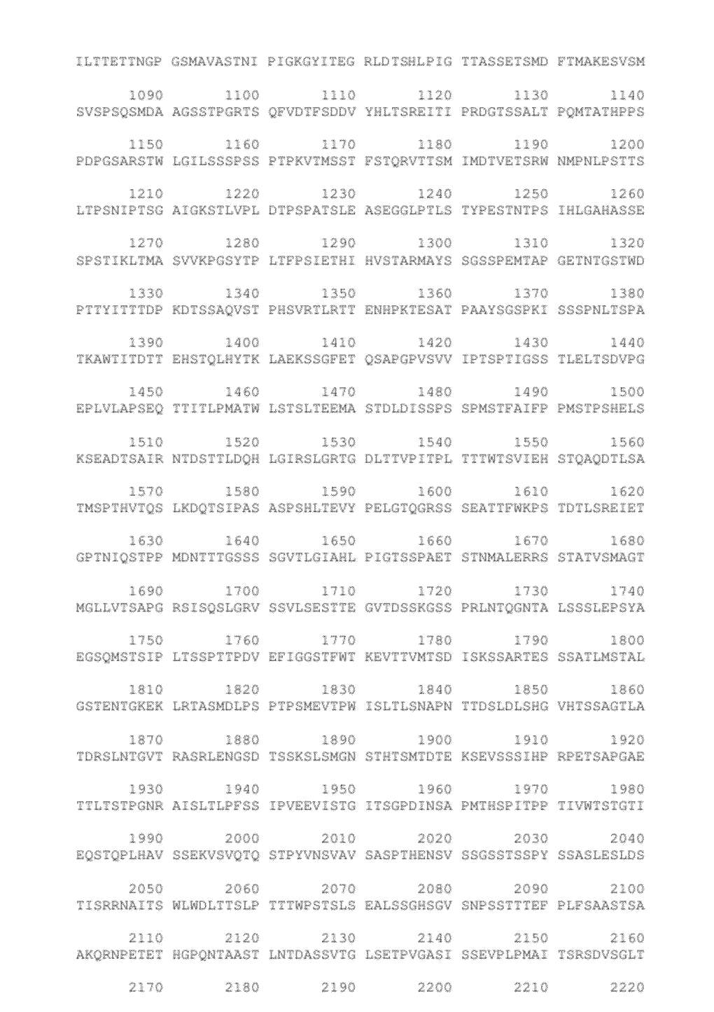

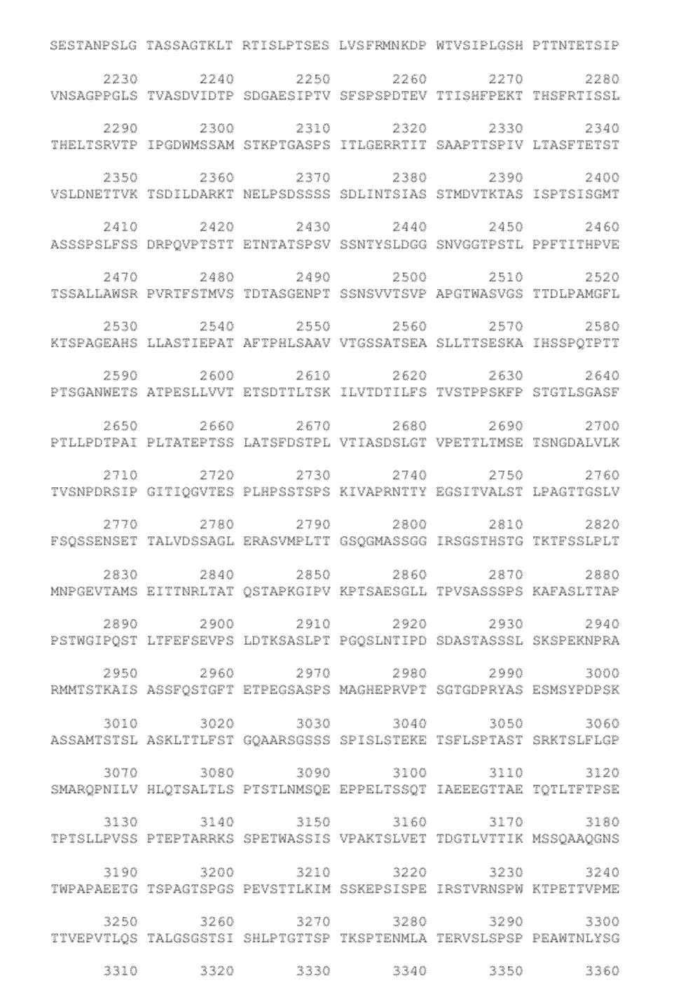

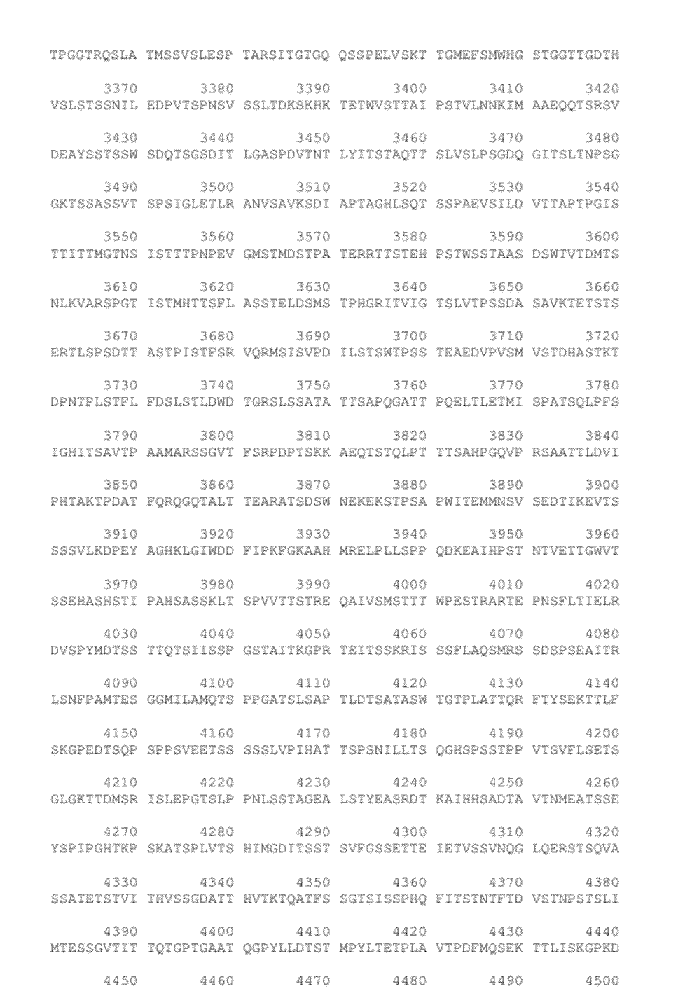

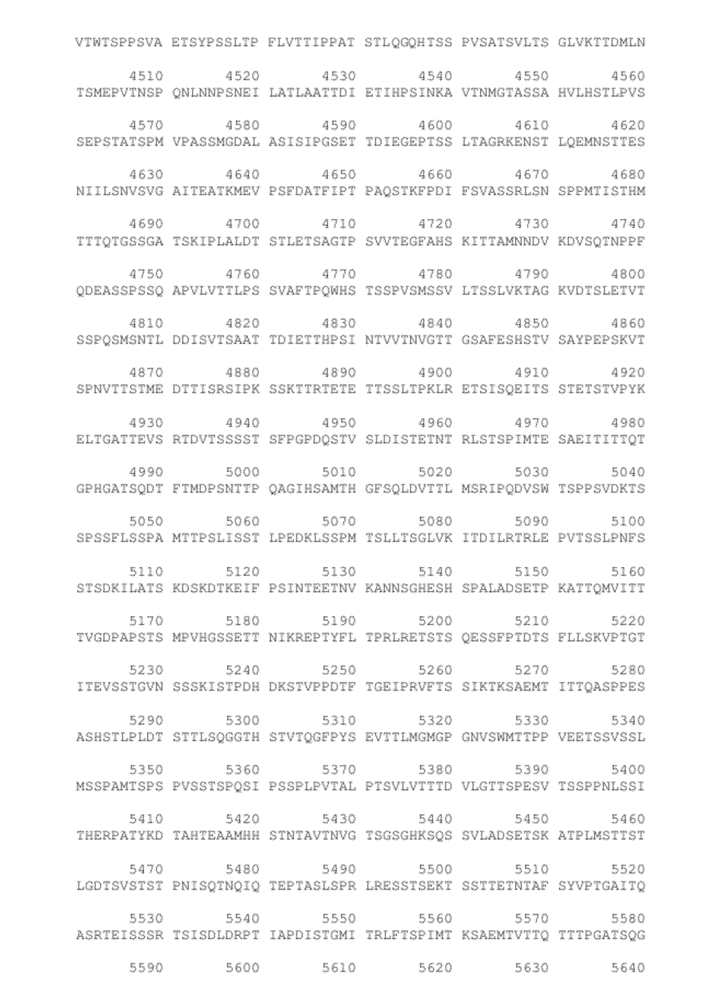

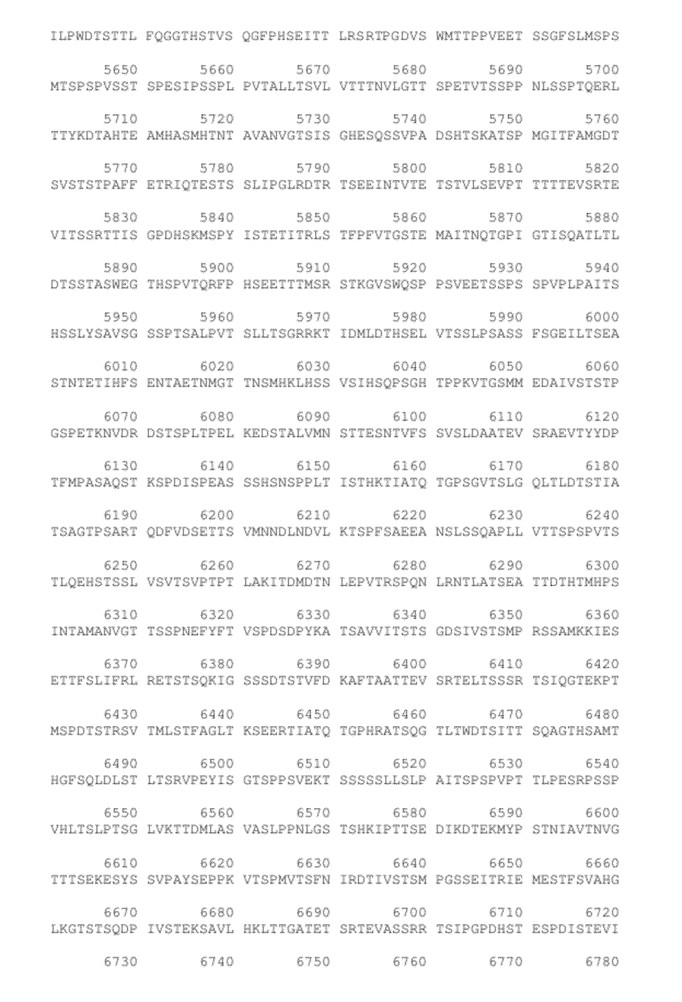

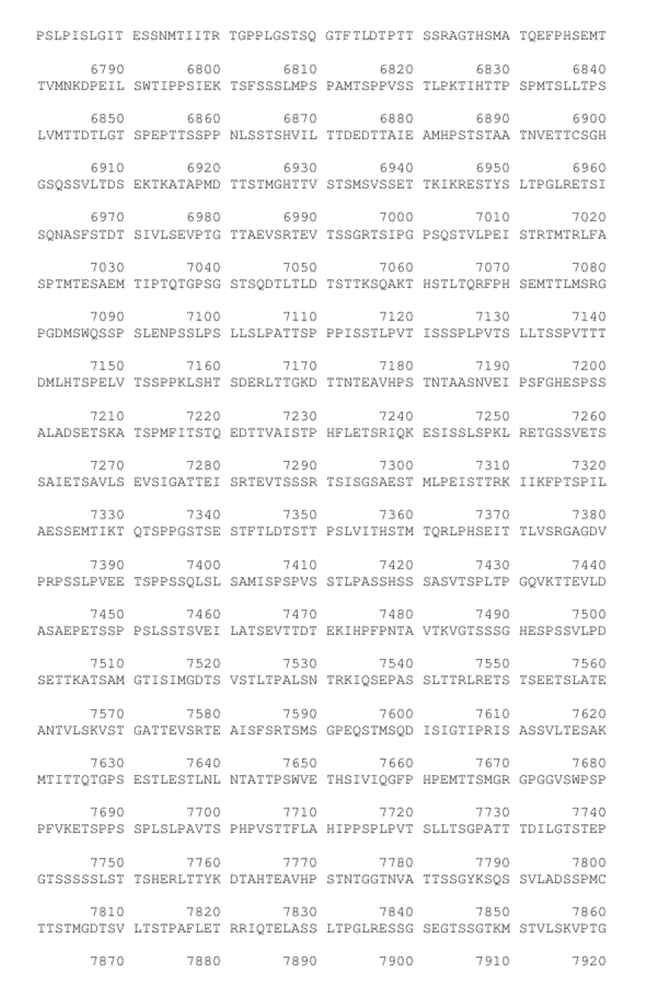

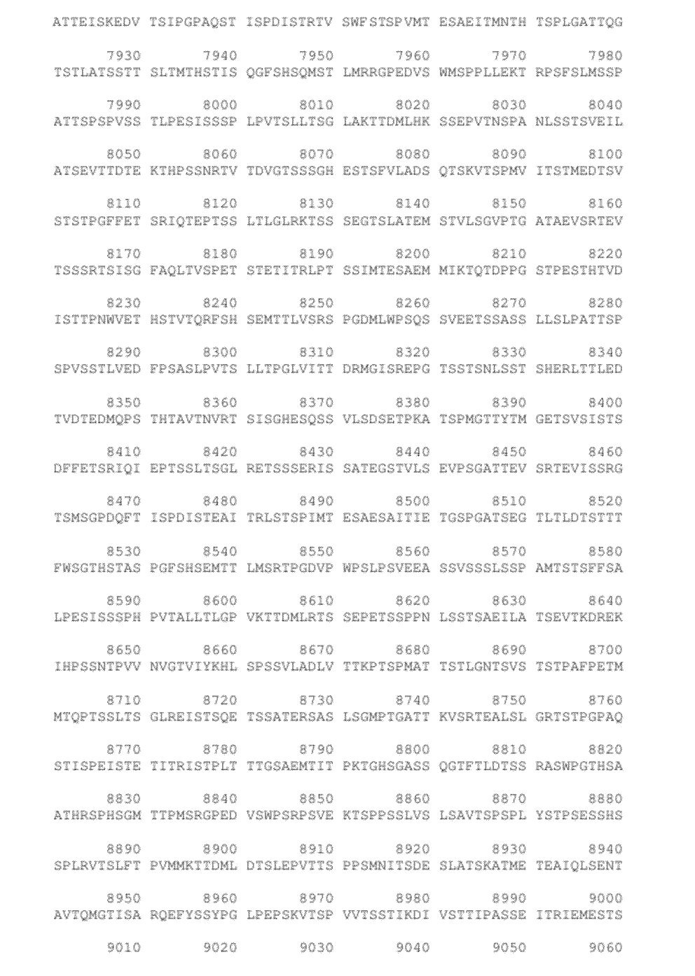

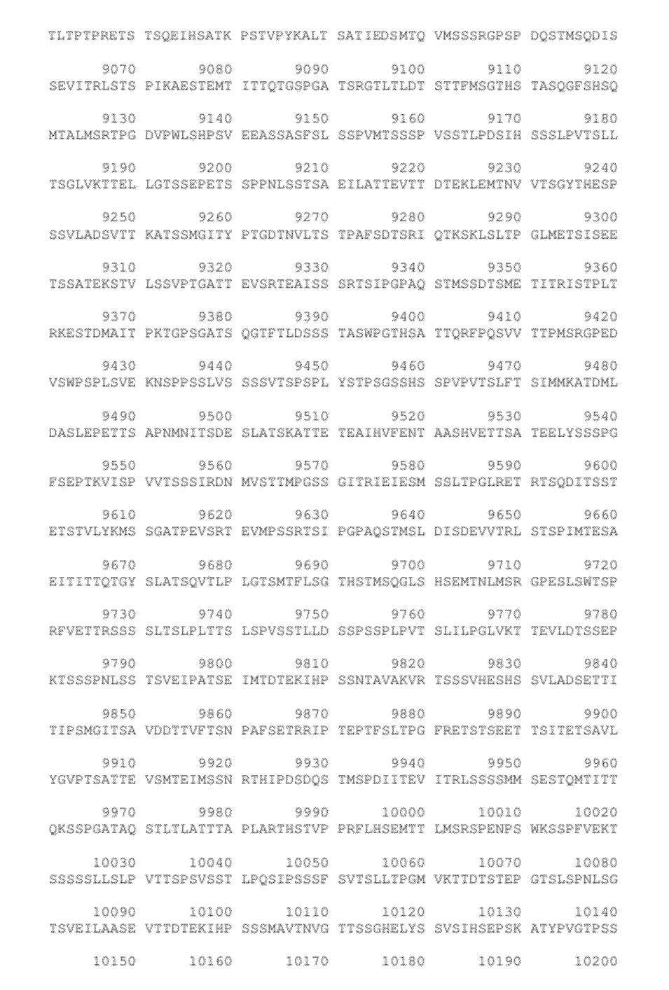

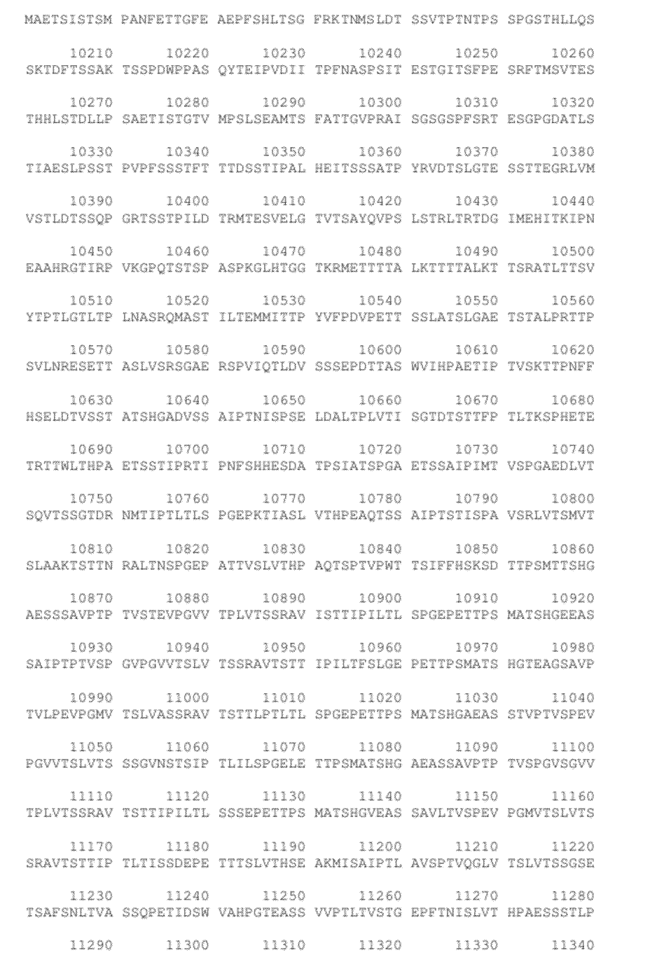

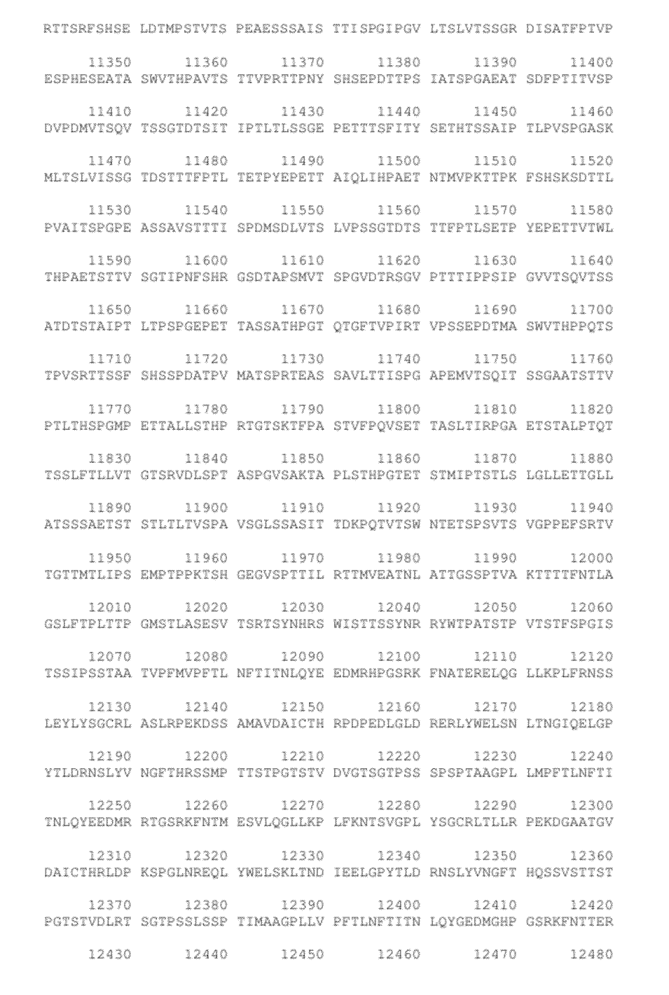

Another exemplary biomarker present in the panel of the invention is CA 125. CA 125 is a 22152 amino acid protein (Swiss-Prot Accession number Q8WXI7). The amino acid sequence of CA 125 is set forth in FIG. 3 (SEQ ID NO: 2). Antibodies to CA 125 can be made using any method well known in the art, or can be purchased from, for example, Santa Cruz Biotechnology, Inc. (Catalog Number sc-52095) (www.scbt.com, Santa Cruz, Calif.). In aspects of the invention, CA 125 is upregulated in subjects with ovarian cancer as compared to subjects that do not have ovarian cancer.

2.4 Transthyretin (Prealbumin)

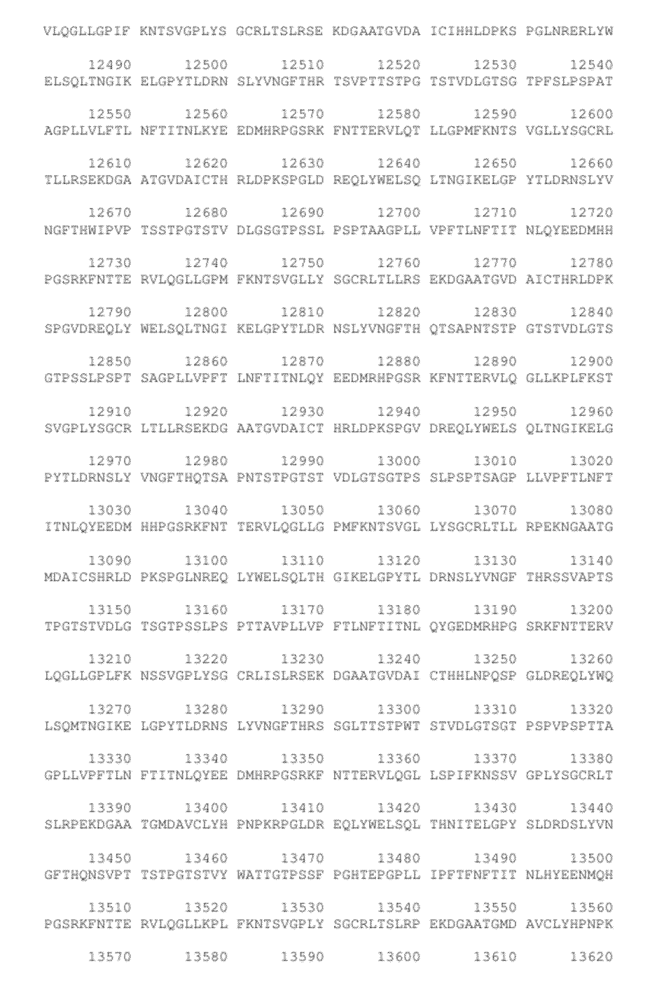

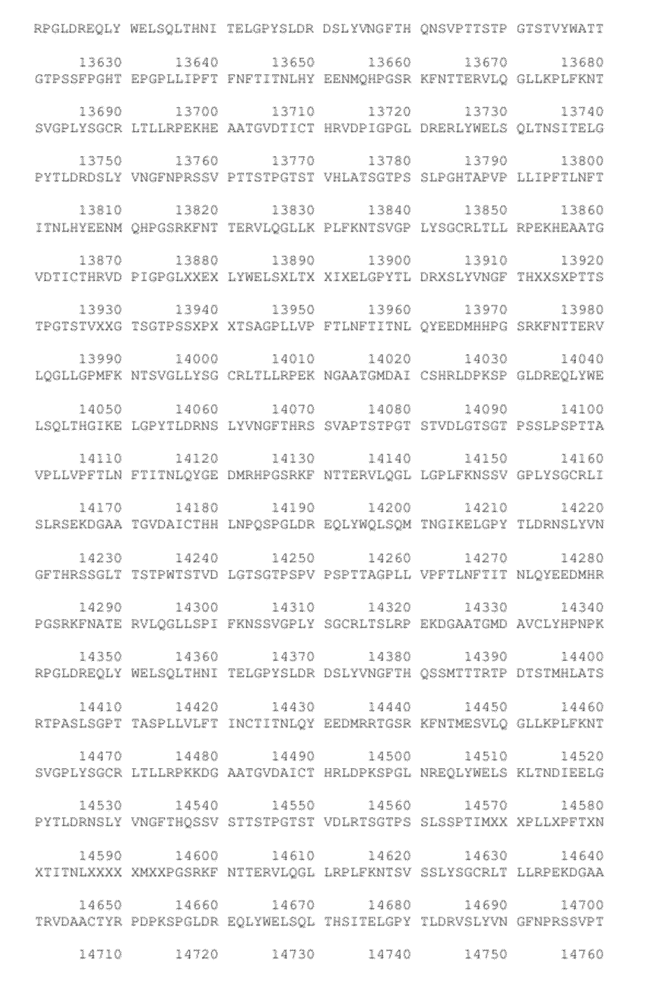

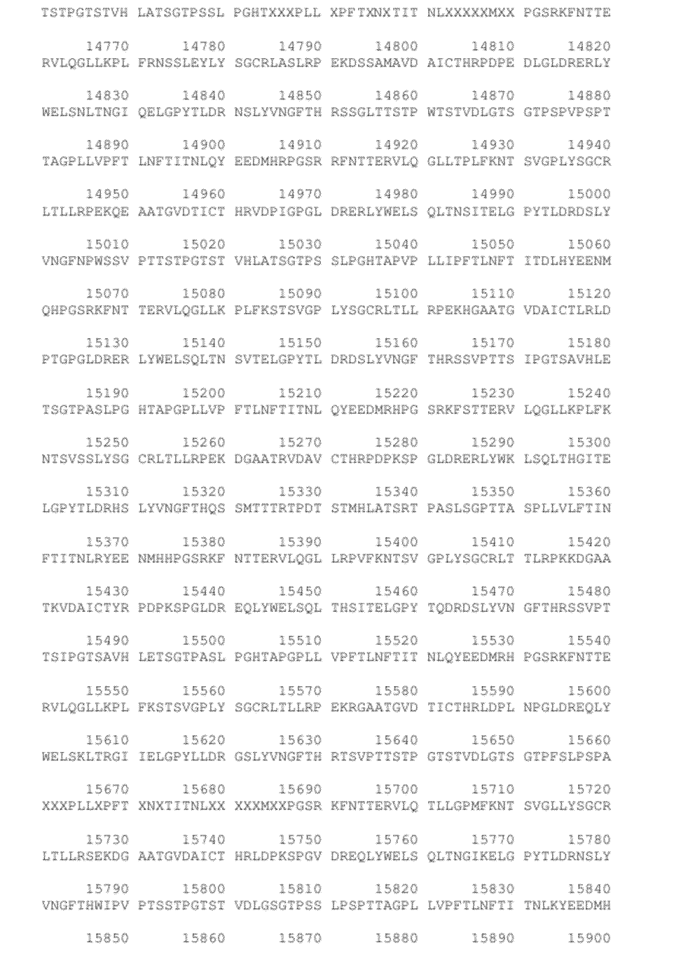

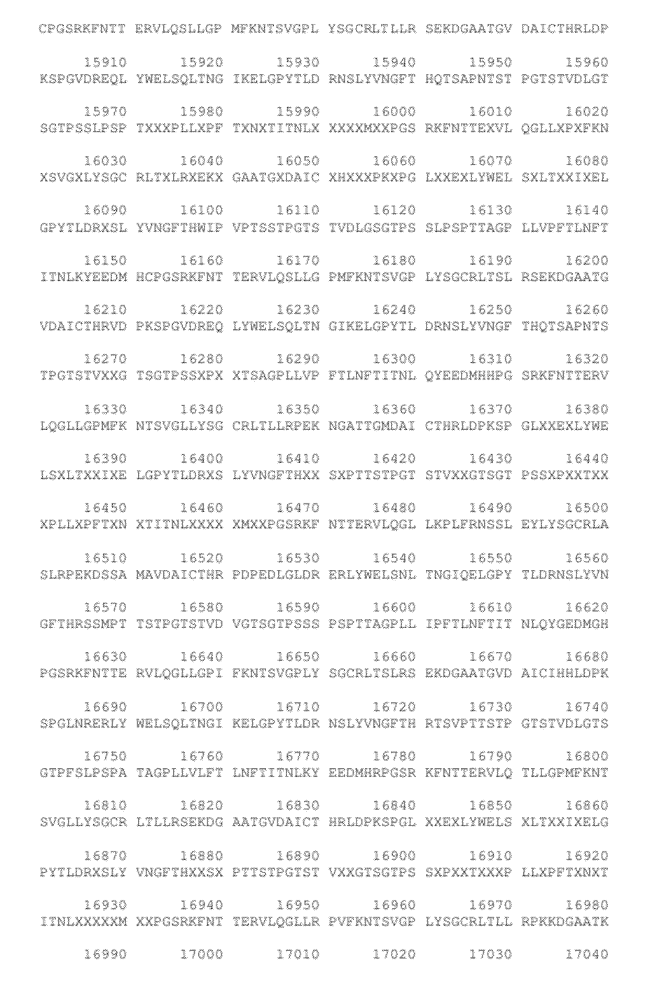

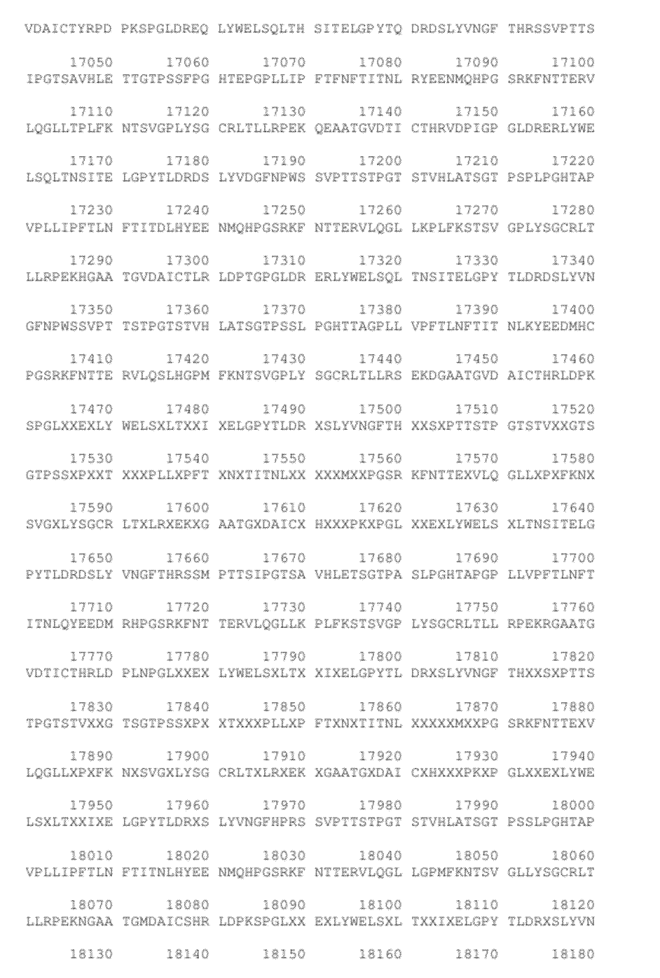

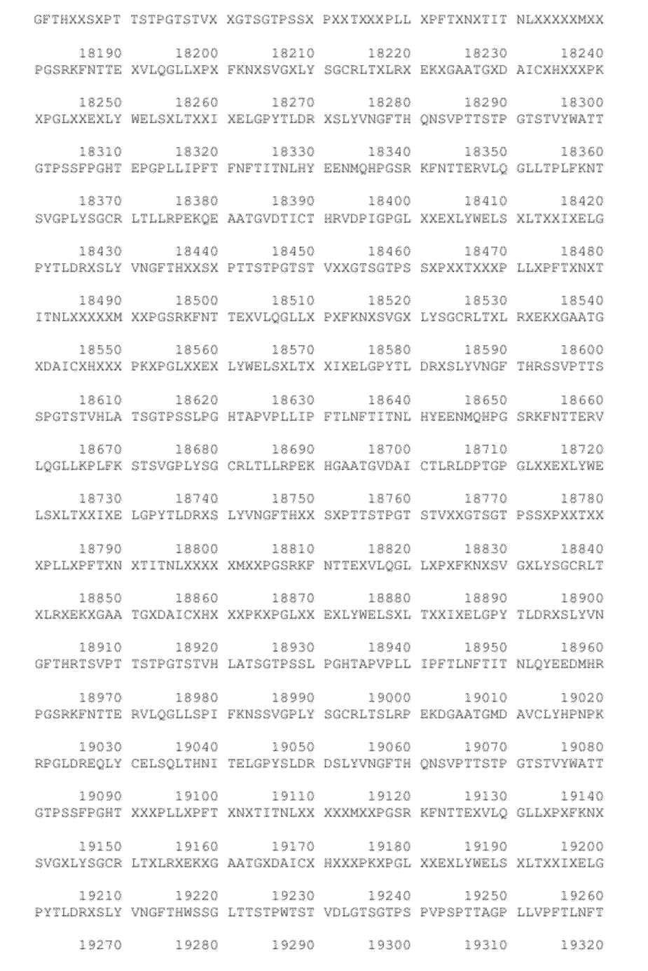

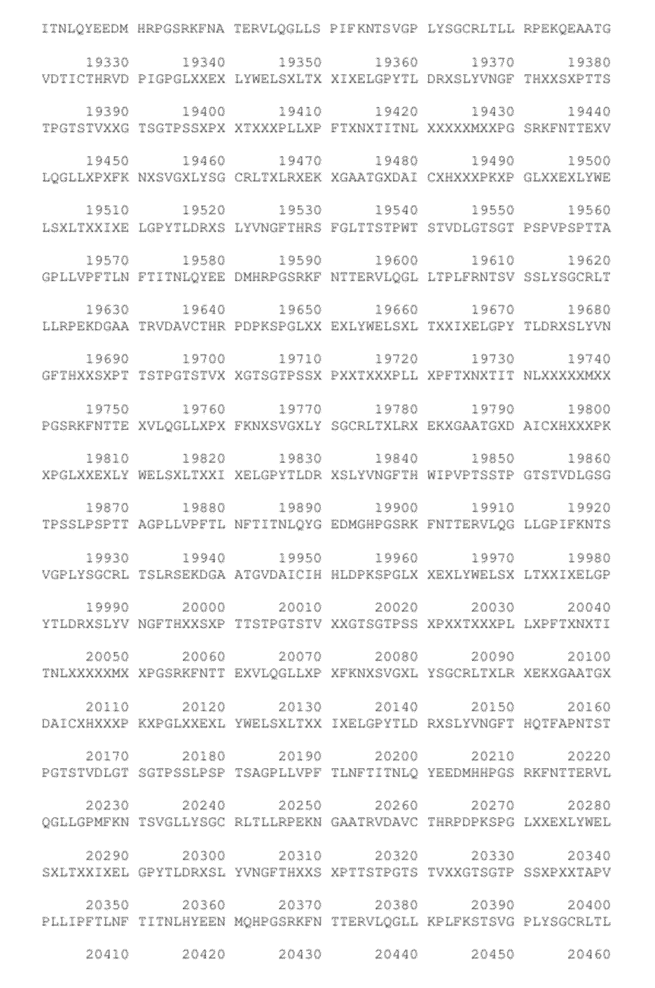

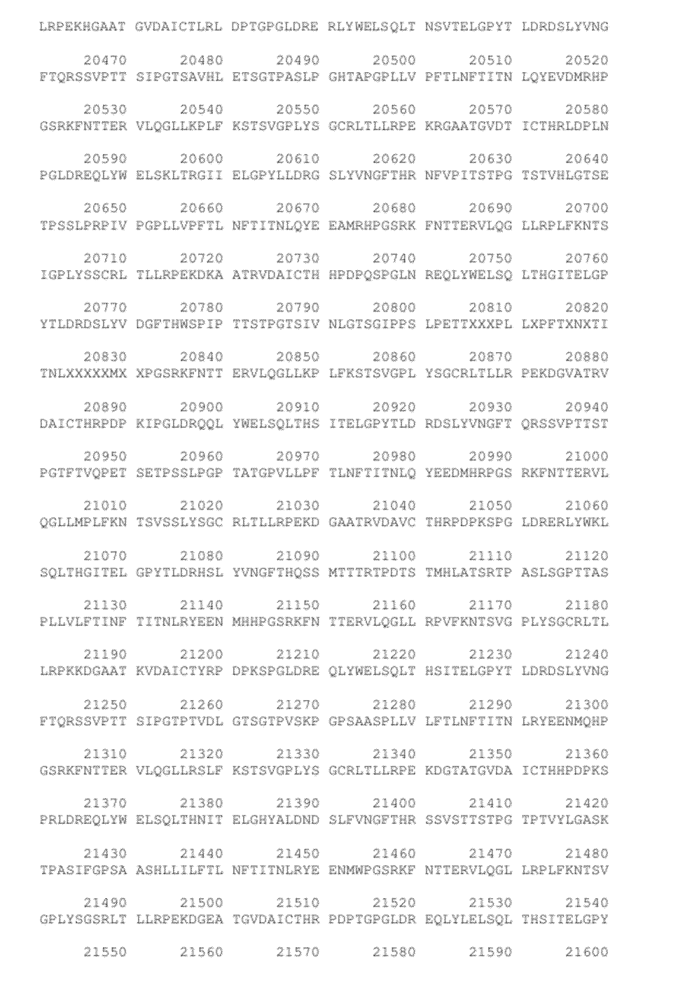

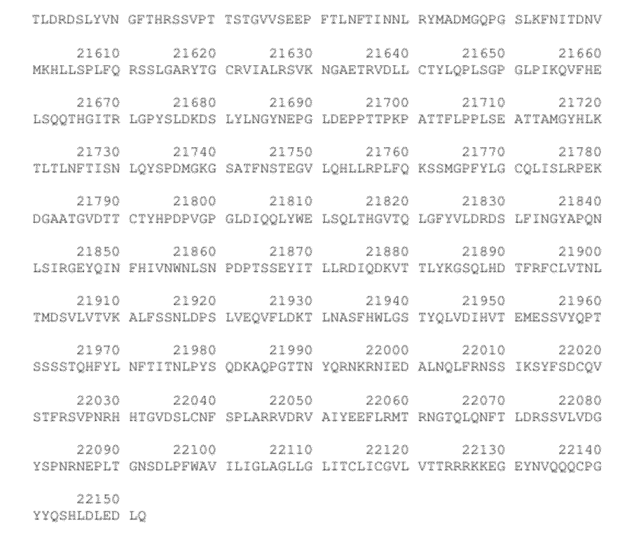

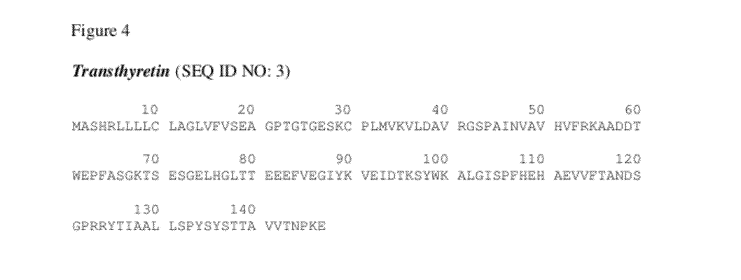

Another exemplary biomarker present in the panel of the invention is a form of pre-albumin, also referred to herein as transthyretin. Transthyretin is a 147 amino acid protein (Swiss Prot Accession number P02766). The amino acid sequence of transthyretin is set forth in FIG. 4 (SEQ ID NO: 3). Antibodies to transthyretin can be made using any method well known in the art, or can be purchased from, for example, Santa Cruz Biotechnology, Inc. (Catalog Number sc-13098) (www.scbt.com, Santa Cruz, Calif.). In aspects of the invention, transthyretin is downregulated in subjects with ovarian cancer as compared to subjects that do not have ovarian cancer.

2.5 Apolipoprotein A1

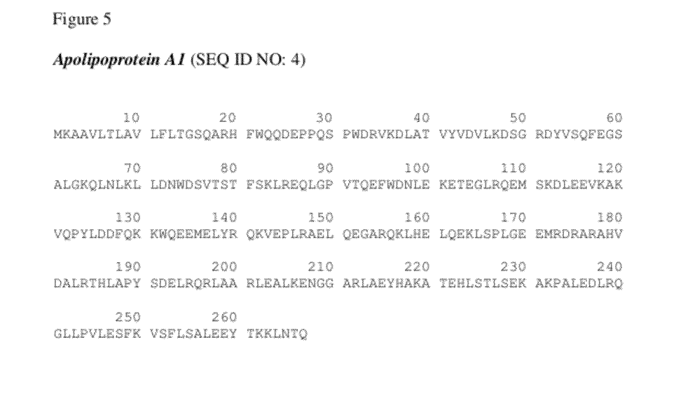

Apolipoprotein A1, also referred to herein as “Apo A1” is another exemplary biomarker in the panel of biomarkers of the invention. Apo A1 is a 267 amino acid protein (Swiss Prot Accession number P02647). The amino acid sequence of Apo A1 is set forth in FIG. 5 (SEQ ID NO: 4). Antibodies to Apolipoprotein A1 can be made using any method well known in the art, or can be purchased from, for example, Santa Cruz Biotechnology, Inc. (Catalog Number sc-130503) (www.scbt.com, Santa Cruz, Calif.). In aspects of the invention, Apo A1 is downregulated in subjects with ovarian cancer as compared to subjects that do not have ovarian cancer.

2.6 Transferrin

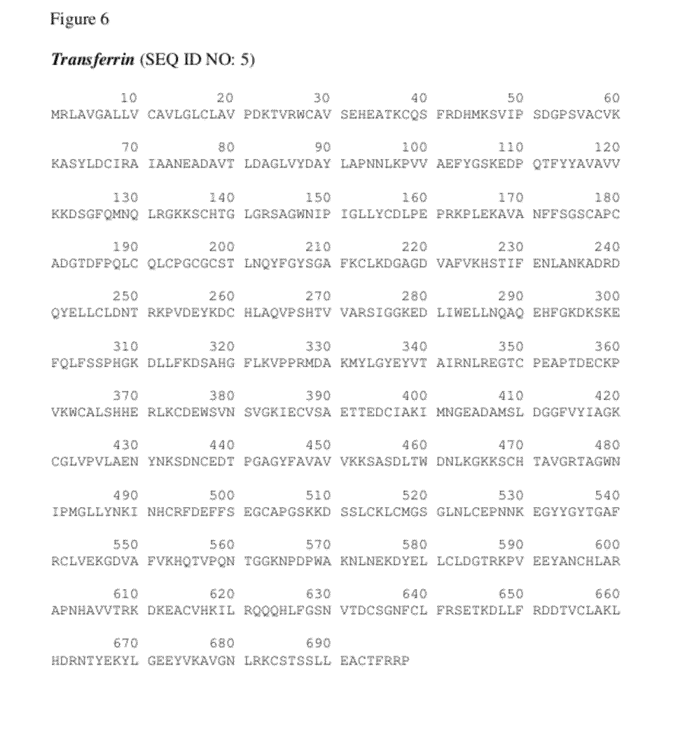

Transferrin is another exemplary biomarker of the panel of biomarkers of the invention. Transferrin is a 698 amino acid protein (UniProtKB/TrEMBL Accession number Q06AH7). The amino acid sequence of transferrin is set forth in FIG. 6 (SEQ ID NO: 5). Antibodies to transferrin can be made using any method well known in the art, or can be purchased from, for example, Santa Cruz Biotechnology, Inc. (Catalog Number sc-52256) (www.scbt.com, Santa Cruz, Calif.). In aspects of the invention, transferrin is downregulated in subjects with ovarian cancer as compared to subjects that do not have ovarian cancer.

3. Biomarkers and Different Forms of a Protein

Proteins frequently exist in a sample in a plurality of different forms. These forms can result from either or both of pre- and post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation. When detecting or measuring a protein in a sample, the ability to differentiate between different forms of a protein depends upon the nature of the difference and the method used to detect or measure. For example, an immunoassay using a monoclonal antibody will detect all forms of a protein containing the epitope and will not distinguish between them. However, a sandwich immunoassay that uses two antibodies directed against different epitopes on a protein will detect all forms of the protein that contain both epitopes and will not detect those forms that contain only one of the epitopes. In diagnostic assays, the inability to distinguish different forms of a protein has little impact when the forms detected by the particular method used are equally good biomarkers as any particular form. However, when a particular form (or a subset of particular forms) of a protein is a better biomarker than the collection of different forms detected together by a particular method, the power of the assay may suffer. In this case, it is useful to employ an assay method that distinguishes between forms of a protein and that specifically detects and measures a desired form or forms of the protein. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology combines mass spectrometry with immunoassay. For example, a biospecific capture reagent (e.g., an antibody, aptamer, Affibody, and the like that recognizes the biomarker and other forms of it) is used to capture the biomarker of interest. In embodiments, the biospecific capture reagent is bound to a solid phase, such as a bead, a plate, a membrane or an array. After unbound materials are washed away, the captured analytes are detected and/or measured by mass spectrometry. (This method will also result in the capture of protein interactors that are bound to the proteins or that are otherwise recognized by antibodies and that, themselves, can be biomarkers.) Various forms of mass spectrometry are useful for detecting the protein forms, including laser desorption approaches, such as traditional MALDI or SELDI, electrospray ionization, and the like.

Thus, when reference is made herein to detecting a particular protein or to measuring the amount of a particular protein, it means detecting and measuring the protein with or without resolving various forms of protein. For example, the step of “detecting β-2 microglobulin” includes measuring β-2 microglobulin by means that do not differentiate between various forms of the protein (e.g., certain immunoassays) as well as by means that differentiate some forms from other forms or that measure a specific form of the protein.

### 4. Detection of Biomarkers for Ovarian Cancer

The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described infra.

4.1. Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

4.2. Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, Calif.), Zyomyx (Hayward, Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

4.3. Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

4.4. Detection by Mass Spectrometry

In aspects of the invention, the biomarkers of this invention are detected by mass spectrometry (MS). Mass spectrometry is a well known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

4.4.1. Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

4.4.2. MALDI and ESI

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using an server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

4.4.3. SELDI

In embodiments, the mass spectrometric technique for use in the invention is “Surface Enhanced Laser Desorption and Ionization” or “SELDI,” as described, for example, in U.S. Pat. No. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate);and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003 -0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

4.5. Detection by Immunoassay

In aspects of the invention, the biomarkers of the invention are measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. Nephelometry is an assay done in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### 5. Methods of the Invention

The biomarkers of the invention can be used in diagnostic tests to assess ovarian cancer status in a subject, e.g., to diagnose ovarian cancer or to determine a course of treatment for a subject. The phrase “ovarian cancer status” includes any distinguishable manifestation of the disease, including non-disease. For example, ovarian cancer status includes, without limitation, the presence or absence of disease (e.g., ovarian cancer v. non-ovarian cancer), the risk of developing disease, the stage of the disease, the progression of disease (e.g., progress of disease or remission of disease over time), the effectiveness or response to treatment of disease, and the determination of whether a pelvic mass is malignant or benign. Based on this status, further procedures may be indicated, including additional diagnostic tests or therapeutic procedures or regimens.

In aspects of the invention, the biomarkers of the invention can be used in diagnostic tests to identify early stage ovarian cancer in a subject.

In aspects of the invention, the biomarkers of the invention can be used in diagnostic tests to select an appropriate course of treatment for a subject diagnosed as being at risk of having ovarian cancer.

The correlation of test results with ovarian cancer involves applying a classification algorithm of some kind to the results to generate the status. The classification algorithm may be as simple as determining whether or not the amounts of the markers listed in Table 1 are above or below a particular cut-off number. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

5.1. Biomarkers

Individual biomarkers are useful diagnostic biomarkers. In addition, as described in the examples, it has been found that a specific combination of biomarkers provides greater predictive value of a particular status than any single biomarker alone, or any other combination of previously identified biomarkers. Specifically, the detection of a plurality of biomarkers in a sample can increase the sensitivity, accuracy and specificity of the test.

Each biomarkers described herein can be differentially present in ovarian cancer, and, therefore, each is individually useful in aiding in the determination of ovarian cancer status. The method involves, first, measuring the selected biomarker in a subject, sample using any method well known in the art, including but not limited to the methods described herein, e.g. capture on a SELDI biochip followed by detection by mass spectrometry and, second, comparing the measurement with a diagnostic amount or cut-off that distinguishes a positive ovarian cancer status from a negative ovarian cancer status. The diagnostic amount represents a measured amount of a biomarker above which or below which a subject is classified as having a particular ovarian cancer status. For example, if the biomarker is up-regulated compared to normal during ovarian cancer, then a measured amount above the diagnostic cutoff provides a diagnosis of ovarian cancer. Alternatively, if the biomarker is down-regulated during ovarian cancer, then a measured amount below the diagnostic cutoff provides a diagnosis of ovarian cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different ovarian cancer statuses, as was done here, and drawing the cut-off to suit the diagnostician's desired levels of specificity and sensitivity.

The biomarkers of this invention (used alone or in combination) show a statistical difference in different ovarian cancer statuses of at least p≦0.05, p≦10⁻², p≦10⁻³, p≦10⁻⁴, or p≦10⁻⁵. Diagnostic tests that use these biomarkers alone or in combination show a sensitivity and specificity of at least 75%, at least 80%, at least 85%, at least 90%, at least 95%, at least 98%, or about 100%.

5.2. Determining Course (Progression/Remission) of Disease

In one embodiment, this invention provides methods for determining the course of disease in a subject. Disease course refers to changes in disease status over time, including disease progression (worsening) and disease regression (improvement). Over time, the amounts or relative amounts (e.g., the pattern) of the biomarkers change. Accordingly, this method involves measuring the panel of biomarkers in a subject at least two different time points, e.g., a first time and a second time, and comparing the change in amounts, if any. The course of disease (e.g., during treatment) is determined based on these comparisons.

5.3. Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the differential presence or absence in a test subject of the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

5.4. Subject Management

In certain embodiments, the methods of the invention involve managing subject treatment based on the status. Such management includes the actions of the physician or clinician subsequent to determining ovarian cancer status. For example, if a physician makes a diagnosis of ovarian cancer, then a certain regime of treatment, such as prescription or administration of therapeutic agent might follow. Alternatively, a diagnosis of non-ovarian cancer or non-ovarian cancer might be followed with further testing to determine a specific disease that might the patient might be suffering from. Also, if the diagnostic test gives an inconclusive result on ovarian cancer status, further tests may be called for.

In one embodiment, the diagnosis may be determining if a pelvic mass is benign or malignant. If the diagnosis is malignant, a gynecologic oncologist may be chosen to perform the surgery. In contrast, if the diagnosis is benign, a general surgeon or a gynecologist may be chosen to perform the surgery.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

### 6. Hardware and Software

The any of the methods described herein, the step of correlating the measurement of the biomarker(s) with ovarian cancer can be performed on general-purpose or specially-programmed hardware or software.

In aspects, the analysis is performed by a software classification algorithm. The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

For example, analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. Exemplary software includes, but is not limited to, Ciphergen's ProteinChip® software, in which data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, for example, as part of Ciphergen's ProteinChip® software package, that can automate the detection of peaks. This software functions by identifying signals having a signal-to-noise ratio above a selected threshold and labeling the mass of the peak at the centroid of the peak signal. In embodiments, many spectra are compared to identify identical peaks present in some selected percentage of the mass spectra. One version of this software clusters all peaks appearing in the various spectra within a defined mass range, and assigns a mass (N/Z) to all the peaks that are near the mid-point of the mass (M/Z) cluster.

In aspects, software used to analyze the data can include code that applies an algorithm to the analysis of the results (e.g., signal to determine whether the signal represents a peak in a signal that corresponds to a biomarker according to the present invention). The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### 7. Generation of Classification Algorithms for Qualifying Ovarian Cancer Status

In some embodiments, data derived from the assays (e.g., ELISA assays) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for ovarian cancer. The classification algorithms, in turn, form the base for diagnostic tests by providing diagnostic values (e.g., cut-off points) for biomarkers used singly or in combination.

### 8. Kits for Detection of Biomarkers for Ovarian Cancer

In another aspect, the invention provides kits for aiding in the diagnosis of ovarian cancer (e.g., identifying ovarian cancer status, detecting ovarian cancer, identifying early stage ovarian cancer, selecting a treatment method for a subject at risk of having ovarian cancer, and the like), which kits are used to detect biomarkers according to the invention. In one embodiment, the kit comprises agents that specifically recognize the biomarkers identified in Table 1. In related embodiments, the agents are antibodies. The kit may contain 1, 2, 3, 4, 5, or more different antibodies that each specifically recognize one of the five biomarkers set forth in Table 1.

In another embodiment, the kit comprises a solid support, such as a chip, a microtiter plate or a bead or resin having capture reagents attached thereon, wherein the capture reagents bind the biomarkers of the invention. Thus, for example, the kits of the present invention can comprise mass spectrometry probes for SELDI, such as ProteinChip® arrays. In the case of biospecific capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for use in any of the methods described herein. In embodiments, the instructions provide suitable operational parameters in the form of a label or separate insert. For example, the instructions may inform a consumer about how to collect the sample, how to wash the probe or the particular biomarkers to be detected.

In yet another embodiment, the kit can comprise one or more containers with controls (e.g., biomarker samples) to be used as standard(s) for calibration.

### EXAMPLES

### Example 1

### The OVA1 Test Improves the Preoperative Assessment of Ovarian Tumors

Objectives: OVA1 is an in vitro diagnostic multivariate index assay (IVDMIA) that combines five immunoassays into a single diagnostic assay. The assay includes measuring the amount of CA 125, transthyretin (prealbumin), apolipoprotein A1, β-2 microglobulin, and transferrin. The instant example evaluates the performance of OVA1 in the preoperative assessment of ovarian tumors. The objective of this study was to evaluate the performance of the OVA1 Test alone, and in conjunction with current clinical parameters, in estimating the risk of malignancy in pre and postmenopausal women scheduled for surgery with an ovarian mass.

Methods: OVA1 was evaluated in women scheduled for surgery for a known ovarian tumor in a prospective, multi-institutional trial involving 27 primary care and specialty sites throughout the United States. Preoperative serum was collected and the OVA1 results were correlated with the physician assessment and surgical pathology. The preoperative malignancy assessment was documented by the enrolling physician after consideration of all available clinical information. Women were excluded from analysis if surgery was not performed, pathology report was not available, or blood specimen was unusable.

Summary of Results: The study enrolled 590 women and 516 were evaluable with a pre-surgical assessment. Fifty two percent were enrolled by non-gynecologic oncologist (non-GO) surgeons. There were 151 ovarian malignancies (29.3%), including: 96 epithelial ovarian cancers (EOC), 9 non-epithelial ovarian malignancies (non-EOC), 28 tumors of low malignant potential (LMP), and 18 malignancies metastatic to the ovary (met). The 235 premenopausal women enrolled (45.5%) accounted for 42 ovarian malignancies. The OVA1 test had the following performance: sensitivity 92.5%, specificity 42.8%, PPV 42.3%, and NPV 92.7%. The OVA1 test significantly improved the clinician's pre-surgical assessment for both non-GO and GO physicians. Sensitivity improved from 72.2% to 91.7% (95% CI=83.0 to 96.1) for non-GO, and 77.5% to 98.9% (95% CI=93.9 to 99.8) for GO. The NPV improved from 89.1% to 93.2% (95% CI=85.9 to 96.8) for non-GO, and 85.5% to 97.6% (95% CI=87.7 to 99.6) for GO. OVA1 correctly identified 70% (non-GO) and 95% (GO) of malignancies missed by the preoperative physician assessment alone. The OVA1 sensitivity by histologic subtype was: EOC 99.0% (95/96), non-EOC 77.8% (7/9), LMP 75.0% (21/28), and met 94.4% (17/18).

Methods

This study was a multi-institutional trial that enrolled patients from 27 primary care and specialty sites throughout the United States. The sites included university and community hospitals, women's health clinics, small obstetrics and gynecology groups, gynecologic oncology practices, and HMO groups. Each participating site obtained approval from their institutional review board. Eligibility criteria included: age 18 years or older, signed informed consent, agreeable to phlebotomy, had a documented ovarian tumor with planned surgical intervention within 3 months of diagnosis, and had no known malignancy in the past 10 years. Women were excluded from analysis if surgery was not performed (27) or delayed more than 3 months (3), pathology report was not available (26), blood specimen was unusable (9), physician assessment was not available (8), or imaging study did not confirm an adnexal tumor (1). Subject demographic and clinicopathological information was collected at each site and recorded centrally.

Preoperative imaging including ultrasound (US), computed tomography scan (CT), or magnetic resonance imaging (MRI) were compulsory to verify an ovarian tumor. All subjects were required to undergo surgery within 3 months of the imaging study. The preoperative assessment was established by the enrolling physician after considering all available clinical information. The physician was asked the following question, “Based on all available clinical information, is the physician of the opinion that this is a malignant ovarian tumor? (yes or no)” Pathology and imaging were systematically reviewed by two independent study physicians. Menopausal status was defined by the absence of menses for at least one year. If the menopausal status was not declared, patients were considered premenopausal when their age was 50 or less, and menopausal when their age was greater than 50.

Preoperative serum was collected by each participating site. 30 to 50 mL of venous blood was collected into BD vacutainer tubes for serum separation (plastic with clot activator, catalog number 367812) and centrifuged after sitting at 18-25° C. for a minimum of 1 hour and maximum of 6 hours post-phlebotomy. Blood was centrifuged at 1200 to 1750× g/RCF (2500-3000 RPM) for 10 to 15 minutes to separate serum from blood cells. The serum specimens for each subject were pooled prior to aliquoting and storage at −65 to −85° C. After preparation, the specimens were shipped frozen to a central biorepository. Specimens were forwarded to one of three OVA1 clinical trial testing sites for biomarker measurements.

The OVA1 Test

The OVA1 test combines five immunoassays into a single diagnostic assay. The five assays are for CA 125, transthyretin (prealbumin), apolipoprotein A1, beta 2 microglobulin, and transferrin. CA 125 was measured on the Elecsys 2010 (Roche) and the other four markers (transthyretin (prealbumin), apolipoprotein A1, beta 2 microglobulin, and transferrin) were measured on the BNII (Siemens). The biomarker assays were conducted according to the manufacturer's directions as detailed in each product insert.

Statistical Methods

The OVA1 statistical analysis stratified data based on physician specialty, menopausal status, stage, and malignant cell type. The cancer prevalence is noted in each table where pertinent. Concordances between OVA1 results of high or low probability of malignancy and pathological findings were assessed using Chi-square (Cramer's V) test. Furthermore, clinically relevant criteria such as sensitivity, specificity, negative predictive value, and positive predictive value were calculated to evaluate the performance of OVA1, preoperative assessment alone, and OVA1 with preoperative assessment. Ninety-five percent confidence intervals (CI) were constructed where appropriate. Statistical analysis was performed with SAS 9.1 (SAS Institute Inc, Cary, N.C.).

Results

The study enrolled 590 women and 516 were evaluable with a pre-surgical assessment. All patients had an imaging study verifying an ovarian mass. Over half of the patients (52%) were enrolled by non-GO surgeons. There were 151 ovarian malignancies (29.3%), including: 96 epithelial ovarian cancers (EOC), 9 non-epithelial ovarian malignancies (non-EOC), 28 tumors of low malignant potential (LMP), 18 malignancies metastatic to the ovary (met). Nine patients with a documented adnexal tumor on imaging study had a pelvic malignancy but normal ovarian histology, and one had both an endometrial cancer and an ovarian tumor of LMP. The mean patient age was 52 (range 18-92). There were 235 (45.5%) premenopausal and 281 (54.5%) postmenopausal women in the evaluable population. The premenopausal women accounted for 42 ovarian malignancies. Benign ovarian conditions were present in 355 women (68.8%). The clinical and histopathological characteristics are summarized in Table 2.

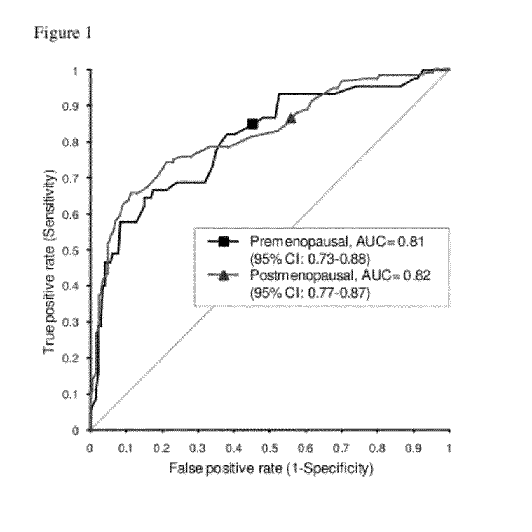

In Table 3, the OVA1 results from malignancy risk assessment are compared to the surgical pathology. The preoperative OVA1 results and the surgical pathology are both significantly and strongly correlated (p<10⁻⁵ and Phi=0.30 for premenopausal women; p<10⁻⁷ and Phi=0.33 for postmenopausal women). The OVA1 test had the following performance: sensitivity 92.5%, specificity 42.8%, PPV 42.3%, and NPV 92.7%. Furthermore, receiver-operating characteristic (ROC) curve analysis also demonstrate a high level of discriminatory power of OVA1 in predicting malignant from benign ovarian tumors, with an area-under-curve of 0.81 (95% CI: 0.73-0.88) and 0.82 (95% CI: 0.77-0.87) for pre- and postmenopausal women, respectively (FIG. 1).

The OVA1 test is intended to provide complementary information in the preoperative risk of malignancy assessment for ovarian tumors. When combined with the clinician's pre-surgical assessment, the OVA1 test shows a consistent improvement in the sensitivity and NPV for both non-GO (Table 4) and GO (Table 5) physicians. As a result, OVA1 correctly identified 70% (non-GO) and 95% (GO) of malignancies missed by the preoperative physician assessment alone. The collective test specificity and PPV decreased when the OVA1 test was added in parallel (and/or) to physician assessment. The OVA1 results remain consistent regardless of menopausal status (Tables 6 and 7). The sensitivity of the OVA1 test by histologic subtype was: EOC 99.0% (95/96), non-EOC 77.8% (7/9), LMP 75.0% (21/28), and met 94.4% (17/18).

The stage distribution for the 105 ovarian malignancies, excluding LMP tumors and non-ovarian cancers, was as follows: 31 stage I, 18 stage II, 51 stage III, and 3 stage IV (stage not available for 2 patients) (Table 8). The OVA1 test maintained high sensitivity regardless of stage. Moreover, for the cancers missed by physician assessment alone, 70% of the primary ovarian cancers were early stage (I or II), and 58% had a normal CA 125 value.

The OVA1 trial was not powered to allow a direct comparison of each individual analyte to the overall OVA1 result; however, it is relevant to consider whether each of the five markers individually contributes to the accuracy of the OVA1 score. The data was analyzed by a nonlinear classifier which makes it difficult to directly calculate the contribution of individual analytes. As an alternative analysis, we replaced the actual values of a single analyte (for all evaluable subjects in the trial) with the analyte's population mean value, and then re-computed a MinusOne result for all the evaluable subjects. We repeated this procedure, one analyte at a time, for all five analytes. Table 9 summarizes the correlations between the OVA1 results and the MinusOne results, and in 2×2 cross-tables compares the corresponding high/low risk assignments. While the overall correlations between OVA1 and the MinusOne results (other than that from missing CA 125) are relatively high, the cross-tables confirm that a significant number of patients, shown in off-diagonal cells, changed risk assignments with each of the missing analytes. This verifies that each of the five analytes individually contributed to the overall OVA1 result for the study population.

Conclusions: The OVA1 test significantly improved sensitivity and correctly identified the majority of patients with ovarian malignancies that were missed by preoperative physician assessment alone. These data support the use of OVA1 in women scheduled for surgery for an ovarian tumor, to facilitate surgical planning, and decisions about referral to a gynecologic oncologist before surgery.

### Incorporation by Reference

All patents, publications, and accession numbers mentioned in this specification are herein incorporated by reference to the same extent as if each independent patent, publication, and accession number was specifically and individually indicated to be incorporated by reference.

TABLE 2
Summary of evaluable subjects

| | All Evaluable | Non-GO | GO |
|---|---|---|---|
| | Subjects | Physicians | Physicians |
| | (N = 516) | (N = 269) | (N = 247) |
| Patient Age, years | | | |
| N | 516 | 269 | 247 |
| Mean (SD) | 52.0 (13.9) | 49.7 (13.6) | 54.6 (13.8) |
| Range (min, max) | 18 to 92 | 19 to 90 | 18 to 92 |
| Menopausal Status, | | | |
| n (%) | | | |
| Pre | 235 (45.5%) | 144 (53.5%) | 91 (36.8%) |
| Post | 281 (54.5%) | 125 (46.5%) | 156 (63.2%) |
| Pathology Diagnosis, | | | |
| n (%) | | | |
| Benign ovarian | 355 (68.8%) | 197 (73.2%) | 158 (64.0%) |
| condition | | | |
| Epithelial ovarian | 96 (18.6%) | 45 (16.7%) | 51 (20.6%) |
| cancer (EOC) | | | |
| Other primary ovarian | 9 (1.7%) | 5 (1.9%) | 4 (1.6%) |
| malignancy (non EOC) | | | |
| Ovarian tumor of low | 28 (5.4%) | 12 (4.5%) | 16 (6.5%) |
| malignant potential | | | |
| (Borderline) | | | |
| Non-ovarian | 18 (3.5%) | 5 (1.9%) | 13 (5.3%) |
| malignancy with | | | |
| involvement of the | | | |
| ovaries | | | |
| Pelvic malignancy | 10 (1.9%) | 5 (1.9%) | 5 (2.0%) |
| with no involvement | | | |
| of ovaries | | | |

TABLE 3
2 × 2 tables comparing OVA1 results for malignancy risk
assessment with primary pathologic determinations

| | OVA1 Result | | |
|---|---|---|---|
| | Low Probability of | High Probability of | |
| Pathology | Malignancy | Malignancy | Row Total |

A. Premenopausal

| | | | |
|---|---|---|---|
| Benign | 98 | 92 | 190 |
| Malignant | 6 | 39 | 45 |
| Column Total | 104 | 131 | 235 |

B. Postmenopausal

| | | | |
|---|---|---|---|
| Benign | 54 | 111 | 165 |
| Malignant | 6 | 110 | 116 |
| Column Total | 60 | 221 | 281 |

TABLE 4
All subjects evaluated by non-GO physicians

| Performance | Preoperative assessment | Preoperative assessment |
|---|---|---|
| non-GO physicians | only | plus OVA1 |
| Sensitivity | 72.2% (52/72) | 91.7% (66/72) |
| 95% CI | 61.0% to 81.2% | 83.0% to 96.1% |
| Specificity | 82.7% (163/197) | 41.6% (82/197) |
| 95% CI | 76.9% to 87.4% | 35.0% to 48.6% |
| PPV | 60.4% (52/86) | 36.5% (66/181) |
| 95% CI | 49.9% to 70.1% | 29.8% to 43.7% |
| NPV | 89.1% (163/183) | 93.2% (82/88) |
| 95% CI | 83.7% to 92.8% | 85.9% to 96.8% |

| | |
|---|---|
| Prevalence | 26.8% (72/269) |

TABLE 5
All subjects evaluated by GO physicians

| Performance | Preoperative assessment | Preoperative assessment |
|---|---|---|
| GO physicians | only | plus OVA1 |
| Sensitivity | 77.5% (69/89) | 98.9% (88/89) |
| 95% CI | 67.8% to 85.0% | 93.9% to 99.8% |
| Specificity | 74.7% (118/158) | 25.9% (41/158) |
| 95% CI | 67.4% to 80.8% | 19.7% to 33.3% |
| PPV | 63.3% (69/109) | 42.9% (88/205) |
| 95% CI | 53.9% to 71.8% | 36.3% to 49.8% |
| NPV | 85.5% (118/138) | 97.6% (41/42) |
| 95% CI | 78.7% to 90.4% | 87.7% to 99.6% |

| | |
|---|---|
| Prevalence | 36.0% (89/247) |

TABLE 6
Premenopausal subjects evaluated by non-GO

| Performance | Preoperative assessment | Preoperative assessment |
|---|---|---|
| Premenopausal | only | plus OVA1 |
| Sensitivity | 65.4% (17/26) | 84.6% (22/26) |
| | 46.2% to 80.6% | 66.5% to 93.8% |
| Specificity | 83.1% (98/118) | 45.8% (54/118) |
| | 75.3% to 88.8% | 37.0% to 54.7% |
| PPV | 45.9% (17/37) | 25.6% (22/86) |
| | 31.0% to 61.6% | 17.5% to 35.7% |
| NPV | 91.6% (98/107) | 93.1% (54/58) |
| | 84.8% to 95.5% | 83.6% to 97.3% |

| | |
|---|---|
| Prevalence | 18.1% (26/144) |

TABLE 7
Postmenopausal subjects evaluated by non-GO

| Performance | Preoperative assessment | Preoperative assessment |
|---|---|---|
| Postmenopausal | Only | plus OVA1 |
| Sensitivity | 76.1% (35/46) | 95.7% (44/46) |
| | 62.1% to 86.1% | 85.5% to 98.8% |
| Specificity | 82.3% (65/79) | 35.4% (28/79) |
| | 72.4% to 89.1% | 25.8% to 46.4% |
| PPV | 71.4% (35/49) | 46.3% (44/95) |
| | 57.6% to 82.2% | 36.6% to 56.3% |
| NPV | 85.5% (65/76) | 93.3% (28/30) |
| | 75.9% to 91.7% | 78.7% to 98.2% |

| | |
|---|---|
| Prevalence | 36.8% (46/125) |

TABLE 8
OVA1 results by cancer stage for primary ovarian malignancies in
all evaluable subjects

| | Stage I | Stage II | Stage III | Stage IV | Not Given |
|---|---|---|---|---|---|
| No. of Subjects* | 31 | 18 | 51 | 3 | 2 |
| Mean (SD) | 6.48 (1.786) | 8.04 (1.596) | 8.26 (1.357) | 8.70 (1.054) | 6.05 (1.626) |
| Median | 6.30 | 8.60 | 8.80 | 8.60 | 6.05 |
| Range | 3.6 to 10.0 | 5.0 to 10.0 | 5.0 to 10.0 | 7.7 to 9.8 | 4.9 to 7.2 |
| OVA1 Positive | 28 | 18 | 51 | 3 | 2 |
| OVA1 Negative | 3 | 0 | 0 | 0 | 0 |
| OVA1 Sensitivity | 90.3% | 100.0% | 100.0% | 100.0% | 100.0% |

*Includes only primary ovarian cancers; LMP tumors and non-ovarian cancers are excluded.

TABLE 9
OVA1 results vs. MinusOne results for individual OVA1
biomarkers

| | MinusOne Neg | MinusOne Pos |
|---|---|---|
| CA 125 | | |
| OVA1 Neg | 0 | 168 |
| OVA1 Pos | 1 | 355 |
| Apolipoprotein A1 | | |
| OVA1 Neg | 159 | 9 |
| OVA1 Pos | 18 | 338 |
| Transthyretin | | |
| (prealbumin) | | |
| OVA1 Neg | 155 | 13 |
| OVA1 Pos | 60 | 296 |
| Beta 2 microglobulin | | |
| OVA1 Neg | 150 | 18 |
| OVA1 Pos | 25 | 331 |
| Transferrin | | |
| OVA1 Neg | 160 | 8 |
| OVA1 Pos | 30 | 326 |

CA 125: Correlation with OVA1 = 0.1690,
Apolipoprotein A1: Correlation with OVA1 = 0.9767,
Transthyretin: Correlation with OVA1 = 0.9389,
Beta 2 microglobulin: Correlation with OVA1 = 0.9743,
Transferrin: Correlation with OVA1 = 0.9695.

What is claimed is:

1. A method for qualifying ovarian cancer status in a subject comprising:
(a) determining the level of biomarkers in a biological sample from the subject, wherein the biomarkers comprise β-2-microglobulin, CA 125, transthyretin (prealbumin), apolipoprotein A1, transferrin, fragments thereof, or a combination thereof; and
(b) comparing the level of the biomarkers to a reference.

2. The method of claim 1, wherein the subject is identified as having ovarian cancer when: i) there is an increase in the amount of β-2-microglobulin or a fragment thereof, ii) there is an increase in the amount of CA 125 or a fragment thereof, iii) there is a decrease in the amount of transthyretin (prealbumin) or a fragment thereof, iv) there is a decrease in the amount of apolipoprotein A1 or a fragment thereof, v) there is a decrease in the amount of transferrin or a fragment thereof relative to the reference, or vi) a combination thereof.

3. The method of claim 1, wherein qualifying ovarian cancer status comprises identifying ovarian cancer in a subject or identifying early stage ovarian cancer in a subject.

4. The method of claim 3, wherein identifying early stage ovarian cancer comprises identifying stage I or stage II ovarian cancer.

5. The method of claim 1, wherein the method further comprises managing subject treatment based on the status.

6. The method of claim 5, wherein the subject is treated with surgery, radiotherapy, chemotherapy, or a combination thereof, if the subject is identified as having ovarian cancer.

7. The method of claim 6, wherein the surgery is performed by a gynecologic oncologist.

8. The method of claim 1, wherein the reference is obtained from i) a patient having ovarian cancer, ii) the subject prior to therapy, or iii) the subject at an earlier time point during therapy.

9. The method of claim 1, wherein the level of the biomarkers is determined by immunoassay, biochip array, nucleic acid biochip array, protein biochip array, mass spectrometry, or a combination thereof.

10. The method of claim 1, wherein the subject is further evaluated by medical imaging, physical exam, laboratory test(s), menopausal status, clinical history, family history, gene test, BRCA test, or a combination thereof.

11. The method of claim 10, wherein the medical imaging comprises ultrasound, computed tomography scan, positron emission tomography, photon emission computerized tomography, magnetic resonance imaging, or a combination thereof.

12. The method of claim 1, wherein the biological sample is blood, plasma, or serum.

13. The method of claim 1, wherein the subject is postmenopausal.

14. The method of claim 1, wherein comparing the level of the biomarkers to a reference is performed by a software classification algorithm.

15. A method for selecting a treatment for a subject diagnosed as being at risk of having ovarian cancer, wherein the method comprises:
(a) determining the level of biomarkers in a biological sample from the subject, wherein the biomarkers comprise β-2-microglobulin, CA 125, transthyretin (prealbumin), apolipoprotein A1, transferrin, fragments thereof, or a combination thereof;
(b) comparing the level of the biomarkers to a reference; and
(c) selecting a treatment selected from the group consisting essentially of: surgery, chemotherapy, radiotherapy, and a combination thereof, wherein the treatment is selected when i) there is an increase in the amount of β-2-microglobulin or a fragment thereof, ii) there is an increase in the amount of CA 125 or a fragment thereof, iii) there is a decrease in the amount of transthyretin (prealbumin) or a fragment thereof, iv) there is a decrease in the amount of apolipoprotein A1 or a fragment thereof, v) there is a decrease in the amount of transferrin or a fragment thereof relative to the reference, or vi) a combination thereof.

16. A kit for aiding the diagnosis of ovarian cancer comprising one or more agents capable of detecting or capturing β-2-microglobulin, CA 125, transthyretin (prealbumin), apolipoprotein A1, transferrin, or a combination thereof.

17. The kit of claim 16, wherein the kit further comprises instructions for using the agent(s) to detect β-2-microglobulin, CA 125, transthyretin (prealbumin), apolipoprotein A1, transferrin, or a combination thereof.

18. The kit of claim 16, wherein the agent(s) comprise an antibody that specifically binds to β-2-microglobulin, CA 125, transthyretin (prealbumin), apolipoprotein A1, transferrin, or a fragment thereof.

19. The kit of claim 16, wherein the kit further comprises one or more control samples.

20. The kit of claim 19, wherein the control sample(s) comprise β-2-microglobulin, CA 125, transthyretin (prealbumin), apolipoprotein A1, transferrin, or a combination thereof.


SEQUENCE LISTING (5 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 22152 aa, Homo sapiens
  MOD_RES (13877)..(13878): Any amino acid
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 3 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 4 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 5 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP